(19) United States
(12) Patent Application Publication
(10) Pub. No.: US 2019/0231472 A1
(43) Pub. Date: Aug. 1, 2019

(54) WEARABLE SURGICAL RECORDING CAMERA

(71) Applicant: VANDERBILT UNIVERSITY, Nashville, TN (US)

(72) Inventors: Alexander LANGERMAN, Nashville, TN (US); William J. RODRIGUEZ, Nashville, TN (US); Eric NOONAN, Wilton, CT (US); Alexander ROED, Charlotte, NC (US); Chris SAVOCA, San Francisco, CA (US)

(21) Appl. No.: 16/260,003

(22) Filed: Jan. 28, 2019

Related U.S. Application Data
(60) Provisional application No. 62/622,482, filed on Jan. 26, 2018.

(51) Int. Cl. A61B 90/00; A61B 90/50

(52) U.S. Cl. CPC A61B 90/361; A61B 90/37; A61B 2090/502; A61B 90/50

(57) ABSTRACT

The present disclosure provides an apparatus for supporting at least one recording device. The apparatus can have a base element, a movable joint, an arm, and a receiving element. The base element can have a horizontal and a vertical segment, where the vertical segment extends from a midpoint along the length of the horizontal segment. The movable joint can connect the arm to the base element. The arm can house the recording device. The receiving element can receive user input. For example, this apparatus can be worn during surgery to video record a surgical procedure.

### CROSS REFERENCE TO RELATED APPLICATIONS

This application claims priority under 35 U.S.C. § 119 to U.S. Provisional Application No. 62/622,482, entitled ‘“CLEOPATRA” WEARABLE SURGICAL VIDEO CAMERA’ and filed on Jan. 26, 2018. The contents of that application are hereby incorporated by reference in their entirety.

### FIELD

The present disclosure relates to an apparatus for supporting a recording device.

### BACKGROUND

Videography in surgery enables scientific and societal benefits by providing objective visual records of surgical proceedings. These records can provide quality/safety review, dissemination of best practices, and documentation for patients' medical records. Monitoring and review of these video records can decrease adverse events in future procedures and can increase safer practices. Videos also provide a prime means of surgical education and are often used in conferences or as online content for surgical textbooks. Surgical video atlases have also been developed as resources by professional surgical groups societies.

However, most conventional videos are from “endoscopic” procedures (e.g., laparoscopy, hysteroscopy, uroscopy, nasal endoscopy, gastrointestinal endoscopy, robotic surgery, or any other surgery where there is no “open” cavity). Endoscopic procedures are enabled through a camera device that the surgeon views while performing the procedure. Therefore, video capture for educational purposes requires no additional technology or devices beyond what is needed for the surgical procedure itself. Endoscopic procedures are a small proportion of all surgical procedures. “Open” procedures are much more common than endoscopic procedures. However, “open” procedures do not require the use of a camera, because the procedure occurs in the surgeon's direct field of vision. Retrieving reliable video footage of these procedures can be extremely rare, and the footage is subject to many quality control issues.

Overhead light- or boom-mounted cameras suffer from obstruction in a field of view from the heads of surgical team members. These cameras do not compensate for the competing brightness from head-mounted light units. Head-mounted cameras, while incident to the surgeon's field of vision, are subject to serious instability and excursions from head movement. Additionally, head-mounted cameras interfere with loupes and risk neck strain from the additional head weight. Neck strain is a serious issue for surgeons as they spend much of their professional work hunched over a surgical area of interest which already exacerbates strain on the surgeon's neck and back. Conventional methods of gaining video footage for “open” procedures can also use sterile, endoscopic cameras. However, these endoscopic cameras typically require an additional team member to operate the camera, and the camera further risks blocking the surgeon's view or access to the surgical area of interest. Additionally, endoscopic cameras carry reprocessing expenses to make the video viable for a viewer.

Furthermore, dedicated videographers can be used in some circumstances, but these videographers are intrusive and can be extremely cost-prohibitive for routine use. Lastly, because lighting has not been optimally integrated into non-endoscopic modalities, open surgical videos have over- or under-exposure that further limits the quality of conventionally-retrieved video footage.

Due to the ease and ubiquitous nature of endoscopic video, substantial progress has been made in endoscopic surgery videos to enhance skills assessment, link quality of technique to outcomes, and use computer vision to automate recognition of anatomy, surgical activity, and skills. “Open” surgical procedures cannot realize the same progress due to the difficulty of capturing reliable, high-quality video of the procedures.

Therefore, what is needed is a system or method of obtaining high-quality video footage for “open” surgical procedures.

### SUMMARY

The present disclosure provides an apparatus configured to support a recording device. The apparatus can include a base element, a movable joint, and a receiving element. The base element can include a horizontal and a vertical segment. The vertical segment can extend at a midpoint along a length of the horizontal segment. The movable joint can connect an arm to the base element. The arm can house the recording device. The receiving element can be configured to receive user input.

In some embodiments, the recording device can include a video recording device or an audio recording device. Moreover, the arm can include a screen, configured to receive visual data from the video recording device and to display the received visual data. In some embodiments, the apparatus can further include an illumination device. The illumination device can be positioned around an exterior edge of the video recording device.

The recording device can include several video recording devices. Each of the video recording devices can be configured to record separately.

In some embodiments, a first video recording device can include a lens of a first shape. A second video recording device can include a lens of a second shape. The first shape can be different from the second shape.

In some embodiments, a sensor can be mounted on the arm. The sensor can include a microphone configured to receive voice commands. In alternative embodiments, the sensor can be mounted on the body. The sensor can include an inertial measurement unit (IMU) sensor or an accelerometer. The IMU can receive rotational orientation data and the accelerometer can receive gravitational force data. In some embodiments, the apparatus can include a strap removably coupled to a first end of the horizontal segment and to a second end of the horizontal segment.

In some embodiments, the apparatus can also include a sensor mounted on the body or the strap. The sensor can be a biological sensor configured to receive physiological data of a user. Biological sensors can include, for example, a heart rate monitor, a blood flow sensor, a sweat sensor, a respiratory rate monitor, a pressure sensor, a blood pressure monitor, a blood glucose level sensor, an electrocardiogram sensor, myoelectric sensor, a skin conductance sensor, or any combination thereof.

The apparatus can also include a coupling element configured to secure the arm against the base element. The coupling element can be a magnet, a clasp, a plurality of fasteners, or a male and female connector element. The arm can include a removable external housing.

In some embodiments, the base element can house a processor, a battery, and a wireless communication module. The processor can be configured to perform any of a series of tasks. For example, the processor can cause that recording device to start and stop recording based on received voice commands, received physiological data of the user, or received user input. The processor can also be configured to aim the illumination device at a target area, based on at least one of: received location data of the target area, received position and orientation data of the arm, and received user input. The location data and the position/orientation data can be received from a sensor mounted on the apparatus. The user input can also be received at the sensor or from the receiving element. The processor can also change a position and/or an orientation of the arm and/or the recording device based on: received location data of the target area, received position and orientation data of the arm, and received user input. The processor can also provide for capturing and storing, at a memory module, a screenshot or video clip from the recording device. The screenshot and/or video clip can be captured and stored based on received user input or data from the recording device.

In some embodiments, the wireless communication module can be communicatively coupled to an external joystick. The processor can be configured to adjust a position and an orientation of the arm based on data received from the external joystick.

In some embodiments, the wireless communication module can be communicatively coupled to an external display and can be configured to send data received from the recording device to the external display.

The wireless communication module can be communicatively coupled to an external computing device. The processor can be configured to send data received from the recording device to the external computing device. The processor can also receive commands from the external computing device that include instructions to adjust a position and/or orientation of the arm or an array of illumination devices on the arm.

In some embodiments, the arm can include an actuator. The actuator can adjust a position and an orientation of the recording device. The adjustment can be made based on received user input or input from a sensor housed on the apparatus. It should be understood “position” can refer to X-Y-Z movement of the apparatus, and “orientation” can refer to a relative angular position of the apparatus.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The accompanying drawings exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1A shows a front perspective of an exemplary apparatus, according to an embodiment of the present disclosure.

FIG. 1B shows a side perspective of the exemplary apparatus of FIG. 1A, according to an embodiment of the present disclosure.

FIG. 2 shows an exploded view of the exemplary apparatus of FIG. 1A, according to an embodiment of the present disclosure.

FIG. 3 shows internal components of the exemplary system, according to an embodiment of the present disclosure.

FIG. 4A shows a view of the components in an exemplary arm, according to an embodiment of the present disclosure.

FIG. 4B shows an exploded view of the arm of FIG. 4A, according to an embodiment of the present disclosure.

FIG. 5A shows an exploded view of an exemplary arm, according to an embodiment of the present disclosure.

FIG. 5B shows a view of the internal components of the arm of FIG. 5A, according to an embodiment of the present disclosure.

FIGS. 6A-6C show exemplary configurations of a plurality of lights on the apparatus, according to an embodiment of the present disclosure.

FIGS. 7A-7E show exemplary camera configurations, according to various embodiments of the present disclosure.

FIG. 8 shows an exemplary sleeve on the arm of the apparatus, according to various embodiments of the present disclosure.

FIGS. 9A-9B show schematic drawings of an exemplary practitioner wearing the apparatus, according to various embodiments of the present disclosure.

FIG. 10 shows an exemplary system, according to an embodiment of the present disclosure.

FIG. 11 shows exemplary stability data, according to various embodiments of the present disclosure.

FIG. 12 shows a schematic block diagram illustrating an exemplary system, in accordance with an implementation of the present disclosure.

### DETAILED DESCRIPTION

The present disclosure is described with reference to the attached figures, where like reference numerals are used throughout the figures to designate similar or equivalent elements. The figures are not drawn to scale, and are provided merely to illustrate the instant invention. Several aspects of the disclosure are described below with reference to example applications for illustration. It should be understood that numerous specific details, relationships, and methods are set forth to provide a full understanding of the invention. One having ordinary skill in the relevant art, however, will readily recognize that the disclosure can be practiced without one or more of the specific details, or with other methods. In other instances, well-known structures or operations are not shown in detail to avoid obscuring the invention. The present disclosure is not limited by the illustrated ordering of acts or events, as some acts may occur in different orders and/or concurrently with other acts or events. Furthermore, not all illustrated acts or events are required to implement a methodology in accordance with the present disclosure.

The present disclosure provides an apparatus for supporting a recording device. The apparatus can include a base element, a movable joint, an arm, and a receiving element. The base element can include horizontal and vertical segments configured to counterweight the arm and provide stability to the recording device. The movable joint can connect the arm to the base element. The arm can house the recording device. The receiving element can receive user input, including the pressing of a button to turn on or off the recording device. For example, this apparatus can be worn during surgery to video record a surgical procedure.

A practitioner can wear the disclosed apparatus around his neck, affixed by a strap. In this configuration, the practitioner can record events without physically holding the recording device; therefore, the disclosed apparatus is particularly advantageous when a user wants to record events, but cannot use his hands during the recording. For example, surgeons, construction workers, athletes, healthcare professionals, and many others, can all use the disclosed apparatus. Although the apparatus is discussed with respect to surgical procedures, it can be used for any recording needs, without limitation.

With the technology described herein, video learning can be extended to open surgery procedures. For open procedures with limited line-of-sight, the disclosed apparatus can enable effective teaching by giving the whole surgical team a view of the region of interest. The disclosed apparatus can also enable development of video reference libraries for uncommon open procedures that trainees may not encounter in residency, and libraries for more common procedures performed by skilled surgeons to disseminate best practices. Open surgical videos can also provide data from real cases for surgical simulation. Audio, video, images, and other data can be transmitted directly to an electronic medical record or other clinical repository for procedure documentation, allowing automated, efficient, and accurate generation of operative reports. Overall, recordings from an apparatus as contemplated herein can allow automated image analysis, video indexing, workflow tracking, and task recognition to open surgical procedures, thereby fulfilling surgical research priorities.

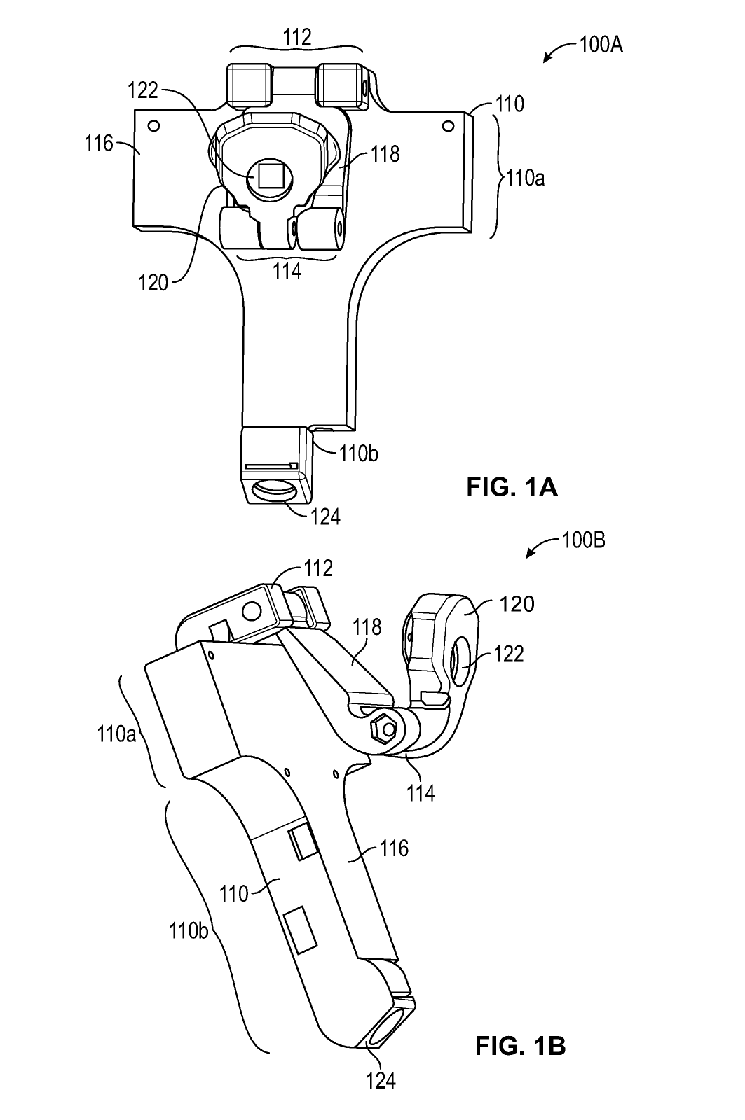

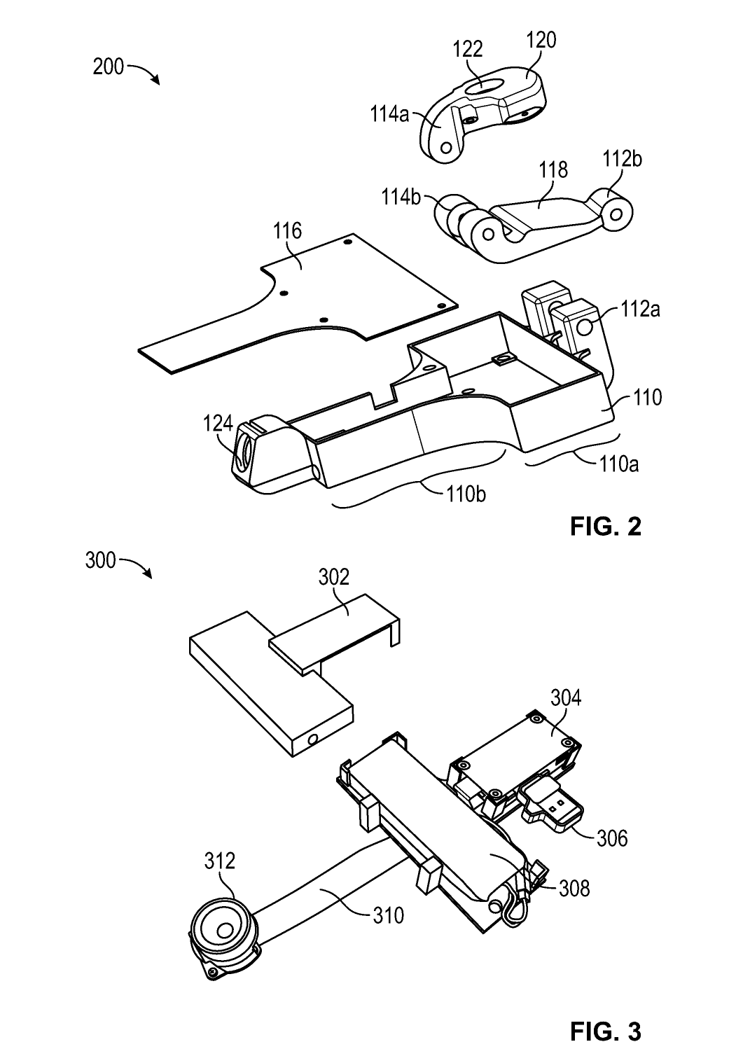

FIGS. 1A, 1B, and 2 show various perspective views of the disclosed apparatus and contain similar elements and reference labels. FIGS. 1A, 1B, and 2 include a base portion 110 with horizontal segment 110a and a vertical segment 110b; a first movable joint 112; a second movable joint 114; a cover 116; a first arm portion 118; a second arm portion 120; an opening 122; and receiving element 124. FIG. 1A shows a front view 100A of the exemplary apparatus; FIG. 1B shows a side view 100B of the exemplary apparatus where the arm portions 118 and 120 can be seen to extend away from the horizontal and vertical segments 110a and 110b; and FIG. 2 shows an exploded view 200 of the same apparatus.

The horizontal segment 110a can provide horizontal stability to the apparatus 100A as the arm portions 118 and 120 extend or retract and as a user bearing apparatus 100A moves around. Vertical segment 110b can further provide a counterweight to apparatus 100A as arm portions 118 and 120 move. Therefore, horizontal and vertical segments 110a and 110b can provide counterweights and stability to a video recording device, if held in the opening 122. In some examples of the present disclosure, the vertical segment 110b can extend from a midpoint along a length of the horizontal segment 110a. Although particular sizes and shapes are shown in FIGS. 1A-2 of the horizontal and vertical segments 110a and 110b, any shapes and sizes of the segments 110a and 110b can be used. Additionally, although vertical segment 110b is shown to have a narrower width than horizontal segment 110a (and horizontal segment 110a has a shorter length than vertical segment 110b), the horizontal and vertical segments can be identically sized and shaped, so as to be indeterminate from each other.

Arm portions 118 and 120 can be attached to the base portion 110 via movable joints 112 and 114, respectively, which can be hinges (with first portions 112a and 114a and second portions 112b and 114b), ball and socket joints, or any other movable joint. Arm portion 120 can contain an opening 122 configured to receive a recording device (not pictured). Any exemplary recording device can be used for the purposes of the present disclosure, including, but not limited to, a camera, a video recording device, a microphone, and/or an audio recording device. Although two arm portions 118 and 120 and movable joints 112 and 114 are shown, any number of arm portions or movable joints can be used, so long as the base portion 110 provides a sufficient counterweight to the arm portions.

The exemplary apparatus can also include a receiving element 124 configured to receive user input. For example, the receiving element 124 can be a button, joystick, switch, sensor, or any other mechanism of receiving user input as known in the art. In some examples, the receiving element 124 can be communicatively coupled to the recording device (not pictured) and can turn on or off the recording device based on received user input. In some examples, the receiving element 124 (particularly when the receiving element 124 is a joystick) can be communicatively coupled to the arm portions 114 and 120 and can automatically adjust the arm portions 114 and 120 based on input from the receiving element 124.

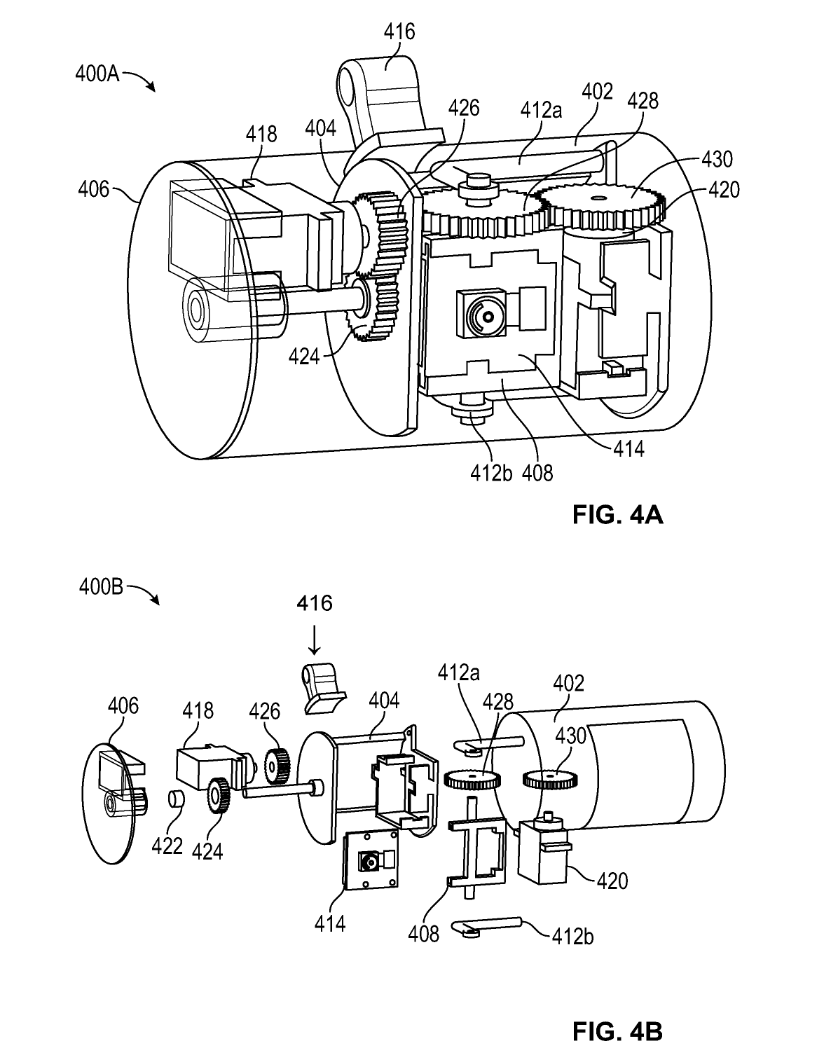

The cover 116 can be a removable cover for the base portion 110; the cover 116 can be opened to reveal a computing system housed within the base portion 110 (for example the system 300 as discussed below with respect to FIG. 3).

In some examples of FIGS. 1A-2, the exemplary apparatus can include a screen (not pictured), mounted on either of the arm portions 118 or 120 or the base portion 110. Such a screen can receive data from the recording device housed in 122 and can display the data. For example, if the recording device is a video, the screen can display captured visual feed data.

In some additional examples of FIGS. 1A-2, the disclosed apparatus can include a sensor, or a plurality of sensors, mounted anywhere on the apparatus, including the arm portions 118 and 120 or the base portion 110. The sensor can be a microphone mounted on the arm to receive voice commands, a physiological sensor mounted on the body to receive biological indicators from a user, or an accelerometer/inertial measurement unit (IMU) sensor. The accelerometer can receive gravitational force data and the IMU sensor can receive rotational orientation data. In some embodiments, the apparatus can turn on or off based on biological indicators from the user. The apparatus can also automatically adjust a position/orientation of the arm portions 114 and/or 120 based on data received from the accelerometer and/or the IMU sensor. This automatic adjustment can provide for automatic stabilization of the recording device.

In some examples, the exemplary apparatus can further include a status indicator light anywhere on the apparatus which identifies whether the recording device is on or off.

FIG. 3 shows internal components of an exemplary system 300, according to an embodiment of the present disclosure. System 300 can include a casing 302; a circuit board 304; an external memory module 306; a battery 308; connectivity cables 310; and a recording device 312. System 300 can be housed within the apparatus 100A of FIGS. 1A-2, for example.

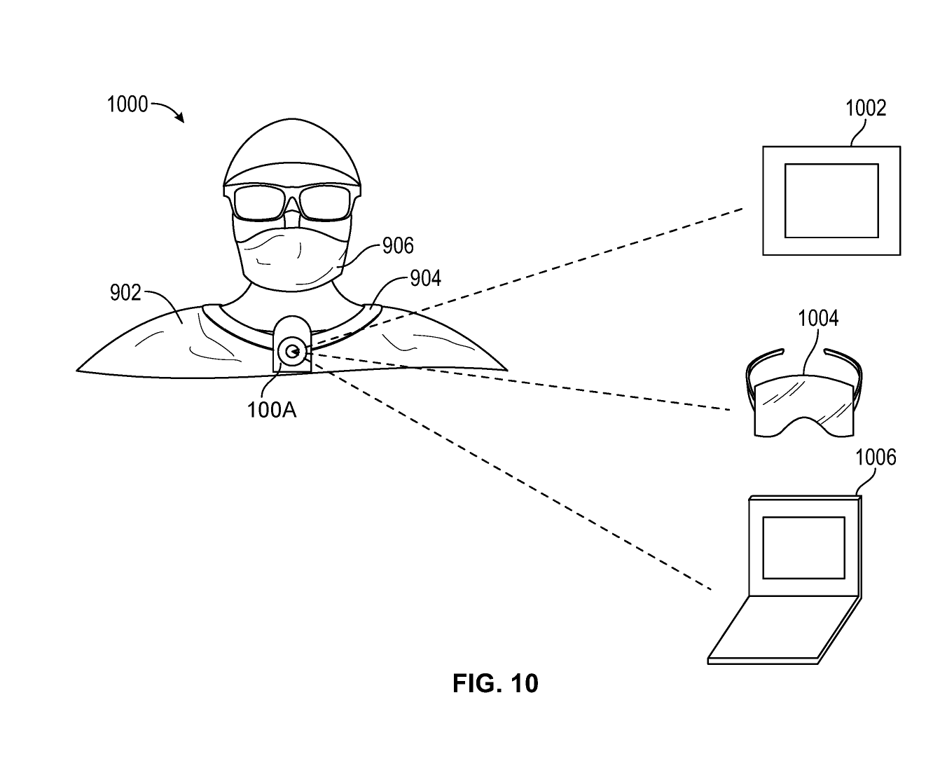

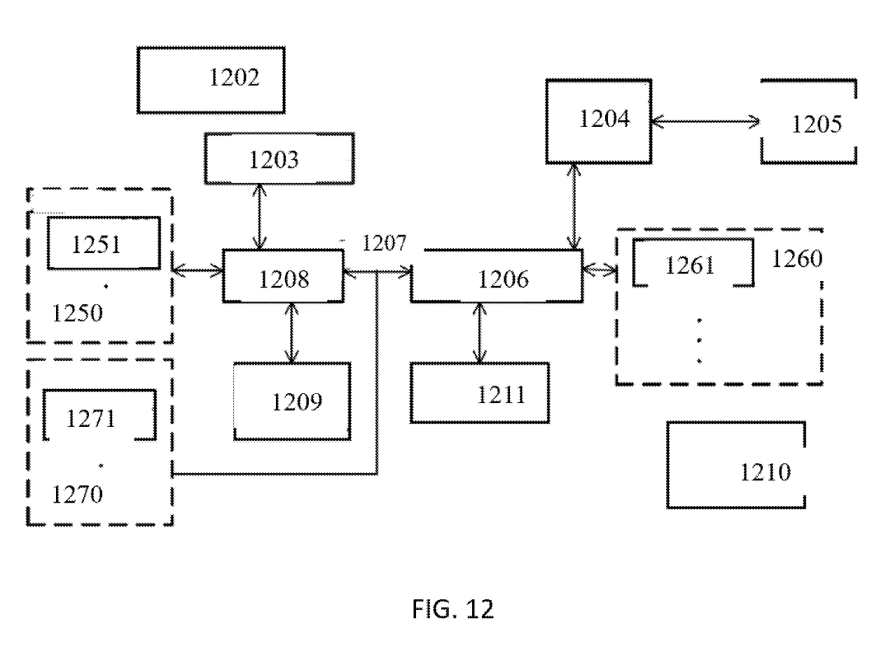

The casing 302 can cover the circuit board 304 and the battery 308. The circuit board 304 can contain a processor, a wireless communication module, and a memory. The wireless communication module can be configured to connect to an external device, for example, via Bluetooth, Wi-Fi, radio frequency, or any other wireless technology without limitation. The processor can send and receive data to an external device via such a wireless communication module (discussed further with respect to FIGS. 10 and 12). The circuit board 304 can further be configured to receive data received from any sensors housed on apparatus 100A.

In some examples, the processor can effect a series of steps based on data received from sensors or an external device (not pictured). Sensors can include any of a microphone, a physiological sensor, a biological sensor, an IMU sensor, a gyroscope, an accelerometer, a light meter, a distance meter, an infrared camera, a visual recording device, and/or a user input device. In some cases, the recording device 312 can be considered a sensor for the purposes of the present disclosure.

For example, the processor can receive any data, including: voice commands from a microphone, physiological data of a user from a biological sensor, image data of a visual feed, instructions from an external device, location data of a target area, position/orientation data of the arm portions and/or recording devices, and/or user input. Based on any of the received data, the processor can (1) cause the recording device 312 to start and stop recording, (2) change a direction or intensity of an illumination device on the apparatus 100A, (3) change a position/orientation of an arm portion 118 or 120, (4) cause an actuator housed in arm portion to move, and/or (4) capture and store, at a memory module, a screenshot or video clip from the at least one recording device.

In some examples, the wireless communication module can be communicatively coupled to an external joystick, and wherein the processor is configured to adjust position and/or orientation of an arm portion based on data received from the external joystick (not pictured). The wireless communication module can also communicatively couple to an external display, and the processor can send data received from the recording device 312 to the external display (not pictured). In some examples, the wireless communication module can be communicatively coupled to an external computing device, and the processor can send data received from the recording device 312 to the external computing device and can receive commands from the external computing device (not pictured).

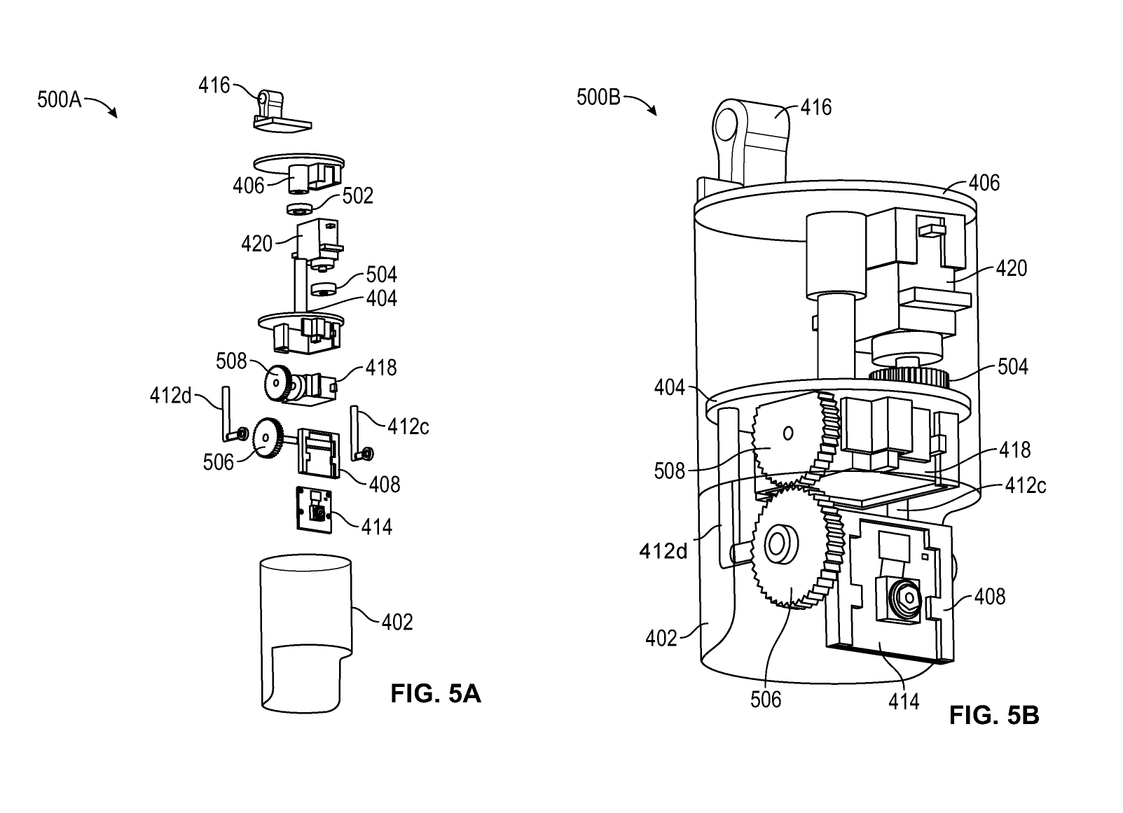

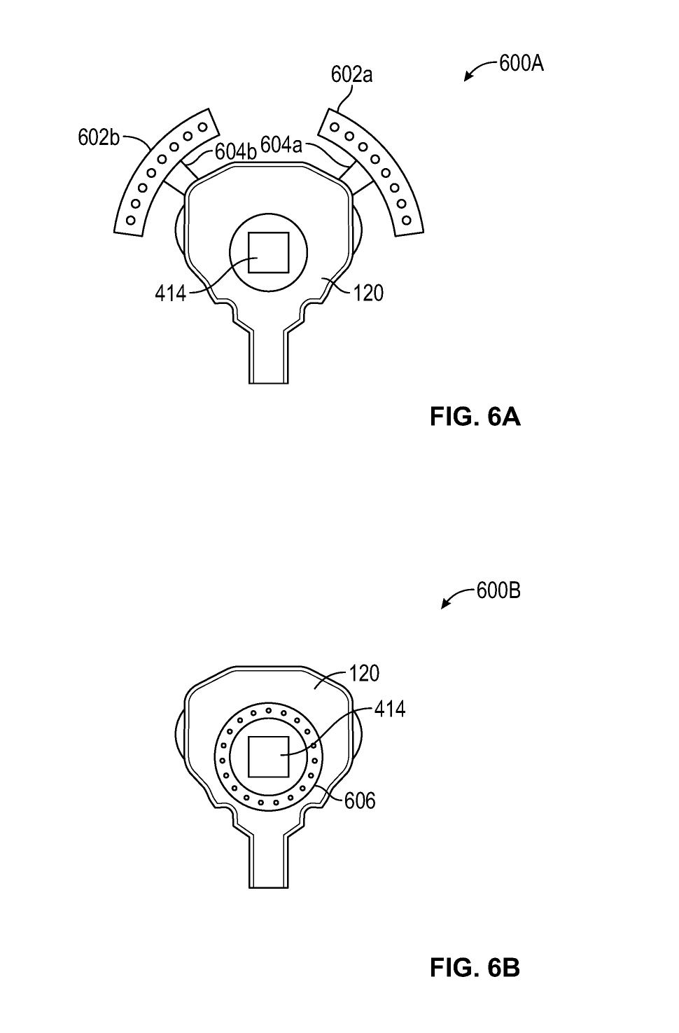

FIGS. 4A-5B show two embodiments of an exemplary arm, according to embodiments of the present disclosure. FIGS. 4A-5B contain similar elements and reference labels, including a case 402; a rotation plate 404; an outer plate 406; a camera clip 408; clip supports 412a and 412b; a recording device 414; a hanger 416; a vertical actuator 418; a horizontal actuator 420; a bearing 422; and gears 424, 426, 428, and 430. In addition, FIGS. 5A-5B include gears 502, 504, 506, 508, and clip supports 412c and 412d. The outer plate 406 locates the axel of the rotation plate 404, and is additionally supported by a shim bearing 422 to maintain a stable axis of rotation.

The case 402 can house all elements of the arms 400A or 500A. The camera clip 408 can secure the recording device 414 along with the clip supports 412a and 412b, which are positioned above and below the camera clip 408 (or clip supports 412c and 412d positioned to the right and left of camera clip 408 for FIGS. 5A-5B). The hanger 416 can allow exemplary arms 400A and 500A to attach to a movable joint (such as movable joints 112 and/or 114 of FIGS. 1A-2).

The vertical actuator 418 and horizontal actuator 420 can adjust a horizontal and vertical position (respectively) of the recording device 414. The vertical actuator 418 in FIGS. 4A and 4B can move gears 424 and 426 which can rotate plate 404 to move a vertical position of recording device 414. The vertical actuator 418 in FIGS. 5A and 5B can move gears 506 and 508 which can rotate plate 404 to move a vertical position of recording device 414. The horizontal actuator 420 in FIGS. 4A and 4B can move gears 428 and 430 which can rotate plate 404 to move a horizontal position of recording device 414. The horizontal actuator 420 in FIGS. 5A and 5B can move gears 502 and 504 which can rotate plate 404 to move a horizontal position of recording device 414. The vertical and horizontal actuators 418 and 420 can be controlled by a processor (such as processor 304 of FIG. 3).

Therefore, FIGS. 4A-5B show exemplary arm portions 400A and 500A which can adjust a position and/or orientation of a recording device 414 based on actuators 418 and 420. FIGS. 4A-4B show an arm portion 400A which is contained in a horizontal case 402, while FIGS. 5A-5B show an arm portion 500A contained in a vertical case 402.

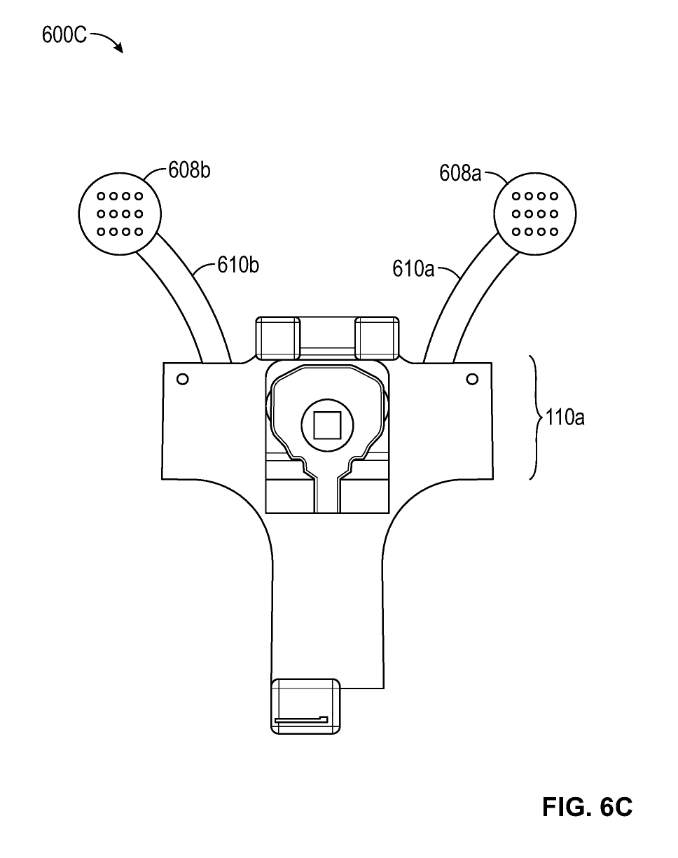

FIGS. 6A-6C show exemplary configurations of a plurality of illumination devices (e.g. lights) on the apparatus, according to an embodiment of the present disclosure. FIG. 6A shows how lights 602a and 602b can be mounted onto the second arm portion 120 by mounting elements 604a and 604b. FIG. 6B shows an apparatus 600B where an array of lights 606 can be positioned around an exterior of a camera 414 on the second arm portion 120. FIG. 6C shows yet another embodiment where lights 608a and 608b can be affixed to the horizontal segment 110a of the apparatus 100A via mounting elements 610a and 610b. The lights can be LED arrays which can have adjusted spectra and brightness depending on user preference or processor inputs.

These apparatuses 600A, 600B, and 600C allow lights 602a, 602b, 606, 608a, and 608b to light up an area of interest that the camera 414 is attempting to record. Lights 602a and 602b of FIG. 6A and lights 608a and 608b of FIG. 6C can be angled, rotated, or moved into different positions via mounting elements 604a and 604b or 610a and 610b, respectively. Such positioning can be effected by communication from a processor (as discussed with respect to FIG. 3) or via manual positioning by a user.

These configurations of lights can provide for consistently lighting an area of interest to increase video quality when the recording device is a camera. Conventional boom-mounted lights are often impaired by the surgical team movement, and head-mounted lights typically contribute to surgeon neck-strain. Lights on the arm portion of an exemplary apparatus can provide unobstructed view of the area of interest without increasing surgeon neck-straining. Although several exemplary lighting configurations are shown in FIGS. 6A-6C, lights can be placed anywhere on the disclosed apparatus, so long as they can face an area of interest.

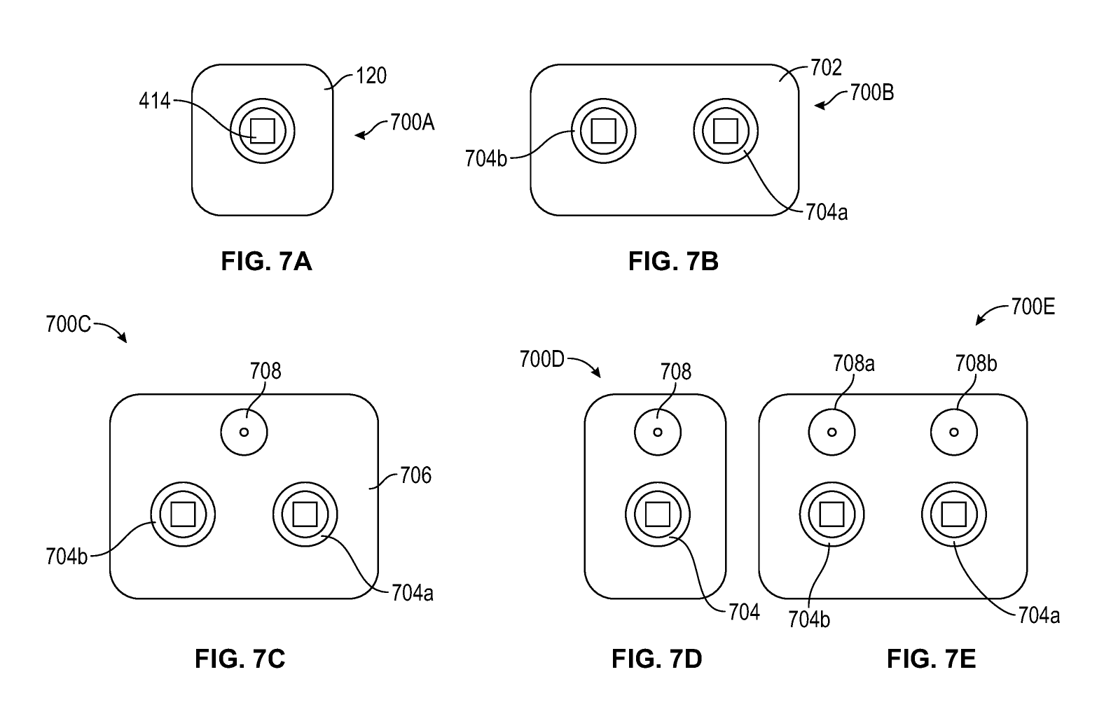

FIGS. 7A-7E show exemplary camera configurations, according to various embodiments of the present disclosure. FIG. 7A shows a camera apparatus 700A similar to FIG. 4A, with an opening 120 and a recording device 414. Apparatus 700A includes a single video camera recording device with a single lens. FIG. 7B shows an apparatus 700B which has two recording devices 704a and 704b on a housing 702. These two recording devices 704a and 704b can be configured to record independently of each other. This is useful in a surgical application where blood splatters, a camera failure, or any other visibility concerns might interfere with the view of one of the recording devices 704a and 704b. In some examples, the recording devices 704a and 704b can have lenses of different shapes to provide binocular vision to support 3-D imaging. Apparatus 700B can facilitate stereoscopic recording, and other analysis techniques, such as depth mapping, tissue deformation, and motion analysis.

FIG. 7C shows an apparatus 700C with three recording devices 704a, 704b, and 708 on a housing 706. Two of the recording devices 704a and 704b can have lenses of a first shape, while the third recording device 708 can have a lens of a second shape. For example, the first shape can be a narrow lens and the second shape can be a wide lens. Therefore, the present disclosure contemplates that the data recorded from the recording devices 704a, 704b, and 708 can provide a variety of viewing angles and frames to capture the same surgical operation.

FIGS. 7D-7E show additional variations of video recording device placement and sizing. FIG. 7D shows an apparatus 700D with two recording devices 704 and 708, one on top of the other. One of the recording devices 704 and 708 can include a narrow lens, and the other can include a wide lens. Both recording devices 704 and 708 can capture the same data with different views of the surgical area of interest. FIG. 7E shows an apparatus 700E with four recording devices 708a, 708b, 704a, and 704b. Apparatus can provide a first row of recording devices 708a and 708b above a second row of recording devices 704a and 704b.

Altogether, FIGS. 7A-7E demonstrate that the present disclosure contemplates a wide range of recording device arrangements on the arm. For example, some arrangements can be used for 3-D imaging, or have lenses specially designed to focus at the optimal surgical working distance (e.g. 30-45 cm from the central neck).

Although specific arrangements are discussed herein, a variety of locations, lens shapes, and number of recording devices can be used in the disclosed apparatus. Additionally, although video recording devices are discussed with respect to the recording devices of FIGS. 7A-7E, a variety of sensors or other recording devices (e.g., cameras, video recorders, microphones, point cloud sensors, infrared sensors, etc.) can be used to attain various benefits.

Configuration 700A is advantageous as it requires the lowest amount of processing power, and can achieve the many applications described herein with a single lens. Configuration 700E, alternatively, provides the most security in redundant recording and directly enables binocular vision with dual lenses each configured for optimal viewing conditions (e.g., an observer can toggle between a wide and narrow view). The spectrum of available configurations allows the apparatus to be configured to the specific task.

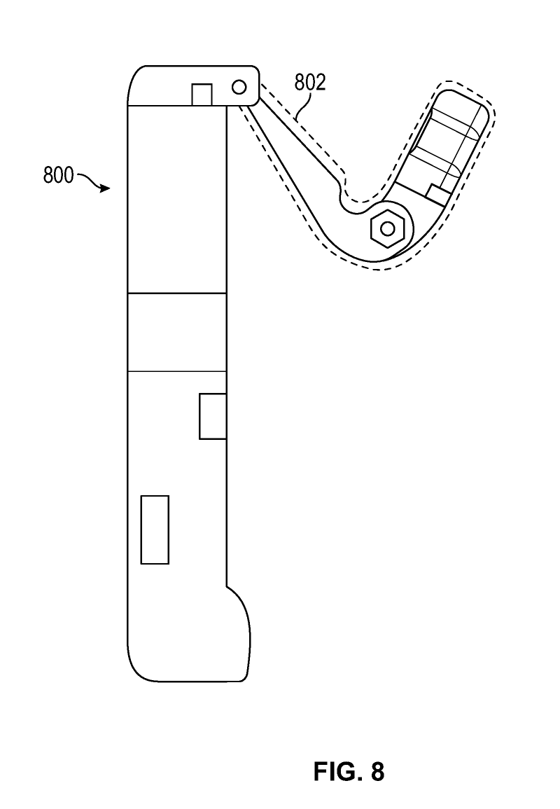

FIG. 8 shows an exemplary sleeve 802 on the arm of the apparatus, according to various embodiments of the present disclosure. In some examples of the present disclosure, the arm portion 118, 120 can have a removable external housing, or a sleeve. A removable external housing 802 can fit around the arm portion 118, 120. In some examples, the removable external housing 802 can be a plastic shell, a cover, or any firm material which can be removed from the apparatus and sterilized in between procedures. In other examples, a removable external housing 802 can include a sleeve, made of a disposable material; such a sleeve can be used for a single procedure, for example, and then disposed of. In either example, the removable external housing 802 can include a first opening, configured to lie adjacent to the base element, and a second opening, configured to leave a space for the at least one recording device, so that the recording device can record input without hindrance. In some embodiments, where the recording device is a video camera, the sleeve can have a transparent material over the second opening so that the recording device can record the visual field of view and the illumination device can illuminate the field without being exposed to contamination. Such a sleeve can be configured specifically to any external arm configuration, examples of which are demonstrated in FIGS. 1B, 4A, 5A, 6, and 7.

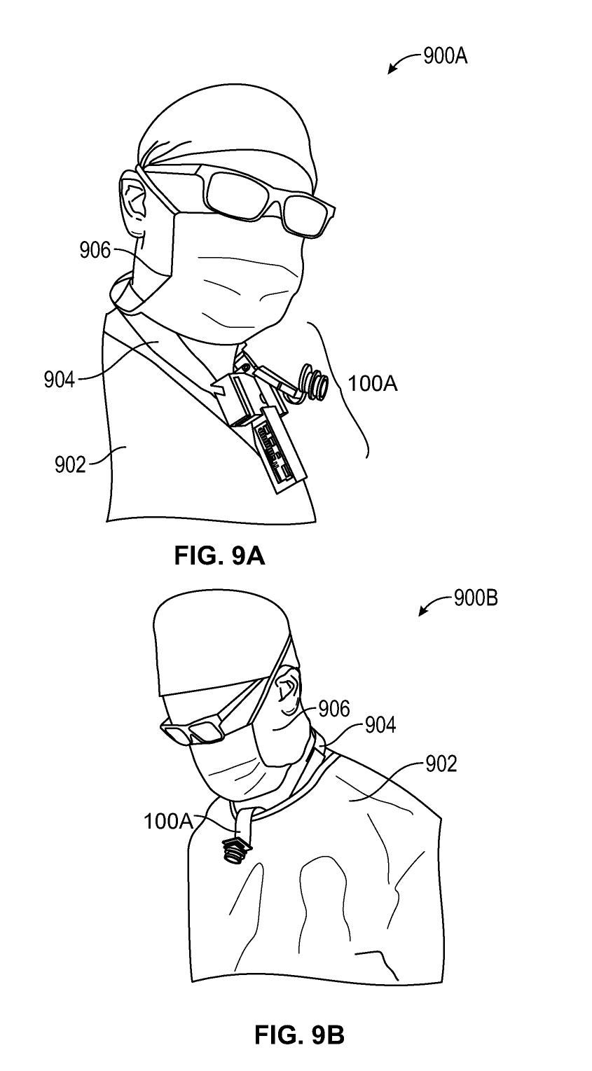

FIGS. 9A-9B show schematic drawings of an exemplary person wearing the apparatus, according to various embodiments of the present disclosure. FIGS. 9A-9B show a user's shirt 902, a strap 904, a user 906, and an apparatus 100A. The apparatus 100A can be as shown and discussed with respect to FIGS. 1A-2. In some embodiments of the present disclosure, the apparatus 100A can be worn by a user 906. The apparatus 100A can include a strap 904 which couples to a first end and a second end of the apparatus 100A. In some instances, the strap 904 can couple to opposing ends of the horizontal segment 110a. The coupling can be temporary to allow the user 906 to remove the strap 904, for example, after a surgical procedure. The present disclosure contemplates that the strap 904 can be coupled to the apparatus 100A by magnets, clasps, mechanical connectors, male and female connectors, or any other means of temporarily affixing the strap 904 to the apparatus 100A. The strap 904 can also be disposable.

FIGS. 9A-9B further demonstrate how the apparatus 100A can be worn in different positions. FIG. 9A shows a first position 900A where the apparatus 100A can be worn entirely outside of a user's shirt 902. For example, the shirt 902 can be a surgical gown. FIG. 9B, shows a second position 900B where the body of the apparatus (the horizontal and vertical segments 110a and 110b) can be placed inside of the shirt 902, while the arm 118 and the recording device are outside of the shirt 902. Position 900B can be particularly useful for surgeons trying to limit contamination during a surgical proceeding. The arm 118 can be sized such that, even if the arm 118 is pointing straight downwards, the arm 118 still lies outside the sterile zone of the gown 902.

In some embodiments, the receiving unit 124 as described with respect to FIGS. 1A-2 can be positioned specifically within a surgical sterile zone, when the apparatus 100A is in position 900B. A surgeon's sterile zone lies below the nipple line of the user 906 of the user's gown/shirt 902. Surgeons are generally not supposed to reach up above the line marking the sterile zone. A length of the vertical segment 110b can be adjusted for each user 906, so that the receiving element 124 will always lie within the user's 906 sterile zone when the apparatus 100A is in position 900B. Position 900B can therefore allow the apparatus 100A be controlled through the sterile portion of the shirt 902 by the user 906. Therefore, apparatus 100A presents considerable advantages over conventional recording devices, because apparatus 100A (1) provides a recording device peaking over the shirt 902 to provide a clear view of the surgical area of interest and (2) can be controlled through the sterile zone by a receiving element, which lies under the shirt 902 and within the sterile zone.

The apparatus 100A can also be secured in position 900B by a coupling element configured to secure the arm 118 against the base element 110. The coupling element can include any of a magnet, a clasp, a plurality of fasteners, a male and a female connector element, or any other coupling agent as readily contemplated by a person skilled in the art. Securing the apparatus 100A in position 900B can hold the apparatus 100A in place on the shirt 902, and can further configure the apparatus 100A to lie in a desirable position on the surgeon. For example, the apparatus 100A can lie along the sternum of the user 906, so as to not interfere with a user's breasts.

The strap 904 can also include one or more sensors. The sensors can be biological sensors configured to receive physiological data of the user 906, including any of a heart rate monitor, a blood flow sensor, a sweat sensor, a respiratory rate monitor, a pressure sensor, a blood pressure monitor, a blood glucose level sensor, an electrocardiogram sensor, a skin conductance sensor, and/or any combination thereof. The sensors can communicatively couple to an internal processor of the apparatus 100A (such as the processor 304 as discussed with respect to FIG. 3) and can provide data to the processor (so that the processor can affect any of the steps discussed above with respect to FIG. 3).

FIG. 10 shows an exemplary system 1000, according to an embodiment of the present disclosure. System 1000 shows how an apparatus 100A can communicatively couple to any of a variety of devices, including an external monitor 1002, a headset 1004, and/or an external computer 1006. The communicative coupling can occur via wires, or any wireless technology as known in the art, including, but not limited to, Bluetooth, Wi-Fi, and radio frequency.

An external monitor 1002 can receive and display visual data received from the apparatus 100A. In some embodiments, the apparatus 100A can communicatively couple to a headset 1004, which can receive and display a visual feed, similar to a virtual reality headset. Such a configuration can be useful, for example, to a surgeon who can look at a monitor 1002 directly in front of him (or at a headset 1004 which the user is wearing) to watch and perform a surgery, instead of hunching over a surgical area of interest. Hunching over a surgical area of interest can injure the surgeon's spine or expose the surgeon's non-sterile zone to the risk of being contaminated (for example, blood splattering onto the surgeon's goggles). In other examples, a user, overseer, or a separate observer can wear the headset 1004 to view the recorded visual feed, including remotely.

The apparatus 100A can also couple with an external computer 1006. The external computer 1006 can receive, display, and/or store any data recorded from the apparatus 100A. In some examples, the external computer 1006 can send instructions to the apparatus 100A, directing the processor to perform any of the steps discussed above with respect to FIG. 3. In some examples, the external computer 1006 (or the screen as discussed above with respect to FIGS. 1A-2) can display light spectrum or step sequence in response to the captured data from the recording device.

Experimental Data

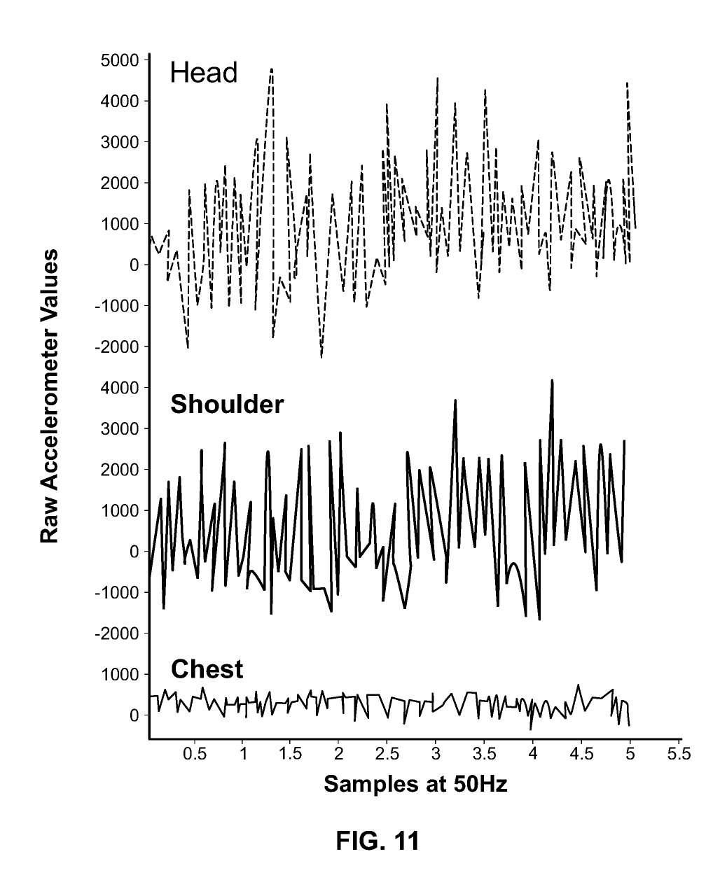

FIG. 11 shows exemplary stability data, according to various embodiments of the present disclosure. FIG. 11 shows data collected from surgeons, where accelerometers were applied to five surgeons during live surgical procedures (at the head, shoulders, and sternum/chest). FIG. 11 shows that mounting a recording device on a user's head or shoulder during a surgical procedure exposes the recording device to a great deal of movement. This movement can interfere with the accuracy or completeness of any data recording. This is especially true for surgeons video recording a surgical procedure, because the camera can move into and out of view of the surgical area of interest during key surgical moments. FIG. 11 demonstrates how a chest-mounted recording device, according to an embodiment of the present disclosure, is exposed to a minimum of movement and can therefore produce an accurate video recording.

Area of Interest Tracking

The present disclosure contemplates that various embodiments of the apparatus can be used to accurately track a surgical area of interest (AOI) with greater success than conventional technology. An apparatus according to the present disclosure can have an uninhibited view of the surgical area of interest throughout the entire surgical procedure and can therefore use various machine learning techniques to continuously track the AOI. An exemplary embodiment of the present disclosure can provide for automatically tracking the AOI and automatically adjusting the position/orientation of the recording device to aim directly at the area of interest, even when the apparatus is being moved around.

An exemplary method of tracking an AOI can include first capturing visual data via at least one camera, and then processing the captured visual data at a processor. A local area of interest can then be determined based on the processed visual data. In some instances, the exemplary method can then provide for determining an orientation for the recording device based on the determined local area of interest and adjusting the recording device to the determined orientation.

In some examples, tracking the AOI can include receiving input from a sensor configured to provide data on an orientation and position of the at least one camera. An exemplary method can then provide active stabilization of the recording device based on the AOI tracking and the received sensor input.

In some examples of the present disclosure, the recording device can automatically capture a screenshot or a video clip based the captured data from the recording device, user input, and/or a determined similarity between the captured visual data and one of a plurality of stored images. For example, the method can provide for searching a plurality of stored images, such as tumor images, and comparing the captured visual data against the plurality of stored images. If a similarity is determined, a screenshot or video clip can be automatically generated.

In some examples, tracking the AOI can include tracking at least one fiducial and capturing visual data based on a location of the at least one fiducial.

Tracking the AOI can further provide for identifying different surgical areas using a range of features. Open surgery images contain three highly-disparate regions (drapes, unperturbed skin, and open cavity). For instance, drapes can be hallmarked by monotonous color and linearity while open cavities can be hallmarked by red tones, irregularity, and depth. Skin and open cavity can be distinguished with color, texture, and depth. During the procedure, hands, instruments, retractors, and sponges enter and leave the field of view, which can be distinguished by color, texture, and depth (white or blue gloves, steel instruments, white sponges). Therefore, in some examples, tracking the AOI can provide for determining a portion of the captured visual data with different coloring from a selected color. The processing can eliminate everything that is not the appropriate coloring and only provide the surgical area.

Such a method of tracking the AOI can be performed in real-time with an exemplary apparatus, during the entire length of a surgical procedure. In some instances, classifiers (such as random forests or convolution neural networks, and any others as known in the art) can be used to localize and track instruments in endoscopic images to assigning pixels to AOI or “other” classes. A very high level of accuracy is not required for this classification step because its main purpose is the localization of the AOI. The recording device focus can be defined as the centroid of the AOI.

An exemplary method can further include inter-frame motion tracking by computing the frame-to-frame displacement of the centroid. Temporal smoothing techniques (e.g. Kalman filtering) can be used to improve the noisiness of the displacement estimate.

In some examples of the present disclosure, a method chosen to track the AOI can be accurate at the beginning of the surgery but become less reliable as the procedure progresses when hands and instruments cover larger portions of the AOI. If this case, an exemplary AOI tracking method can provide for tracking individual points in the image and estimating the AOI displacement from these. Speeded-up robust features (SURF) can be used to detect keypoints with salient features in each frame. Homologous keypoints can be identified between frames. Once homologous keypoints have been identified, inter-frame displacements can be computed.

Accurately tracking the AOI can provide for properly determining lighting needs of the AOI. An exemplary method can provide for correctly exposing the AOI, even if it implies sub-optimal exposure of regions outside of the AOI. The present disclosure can provide for multi-zone metering to determine the optimal exposure. In this approach, light intensity measured at several points is combined using algorithms that are camera-dependent and proprietary. To produce acceptable exposures, the present disclosure can provide for adapting zones to image content (e.g., skin, open cavity, drapes, etc.) and, rather than defining zones as fixed portions of the image as is done in commercial cameras. The exposure can be selected to optimize, for instance, the appearance of the open cavity.

Computer System

FIG. 12 illustrates an exemplary system 1200 that includes a general-purpose computing device 1200, including a processing unit (CPU or processor) 1220 and a system bus 1210 that couples various system components including the system memory 1230, such as read only memory (ROM) 1240, and random access memory (RAM) 1250 to the processor 1220. The system 1200 can include a cache of high speed memory connected directly with, in close proximity to, or integrated as part of the processor 1220. The system 1200 copies data from the memory 1230 and/or the storage device 1260 to the cache for quick access by the processor 1220. In this way, the cache provides a performance boost that avoids processor 1220 delays while waiting for data. These and other modules can control or be configured to control the processor 1220 to perform various actions. Other system memory 1230 may be available for use as well. The memory 1230 can include multiple different types of memory with different performance characteristics. It can be appreciated that the disclosure may operate on a computing device 1200 with more than one processor 1220 or on a group or cluster of computing devices networked together to provide greater processing capability. The processor 1220 can include any general purpose processor and a hardware module or software module, such as module 1 1262, module 2 1264, and module 3 1266 stored in storage device 1260, configured to control the processor 1220 as well as a special-purpose processor where software instructions are incorporated into the actual processor design. The processor 1220 may essentially be a completely self-contained computing system, containing multiple cores or processors, a bus, memory controller, cache, etc. A multi-core processor may be symmetric or asymmetric.

The system bus 1210 may be any of several types of bus structures including a memory bus or memory controller, a peripheral bus, and a local bus using any of a variety of bus architectures. A basic input/output (BIOS) stored in ROM 1240 or the like, may provide the basic routine that helps to transfer information between elements within the computing device 1200, such as during start-up. The computing device 1200 further includes storage devices 1260 such as a hard disk drive, a magnetic disk drive, an optical disk drive, tape drive or the like. The storage device 1260 can include software modules MOD1 1262, MOD2 1264, MOD3 1266 for controlling the processor 1220. Other hardware or software modules are contemplated. The storage device 1260 is connected to the system bus 1210 by a drive interface. The drives and the associated computer-readable storage media provide nonvolatile storage of computer readable instructions, data structures, program modules and other data for the computing device 1200. In one aspect, a hardware module that performs a particular function includes the software component stored in a non-transitory computer-readable medium in connection with the necessary hardware components, such as the processor 1220, bus 1210, output device 1270, and so forth, to carry out the function. The basic components are known to those of skill in the art and appropriate variations are contemplated depending on the type of device, such as whether the device 1200 is a small, handheld computing device, a desktop computer, or a computer server.

Although the exemplary embodiment described herein employs a hard disk as storage device 1260, it should be appreciated by those skilled in the art that other types of computer-readable media which can store data that are accessible by a computer, such as magnetic cassettes, flash memory cards, digital versatile disks, cartridges, random access memories (RAMs) 1250, read only memory (ROM) 1240, a cable or wireless signal containing a bit stream and the like, may also be used in the exemplary operating environment. Non-transitory computer-readable storage media expressly exclude media such as energy, carrier signals, electromagnetic waves, and signals per se. However, non-transitory computer-readable storage media do include computer-readable storage media that store data only for short periods of time and/or only in the presence of power (e.g., register memory, processor cache, and Random Access Memory (RAM) devices).

To enable user interaction with the computing device 1200, an input device 1290 represents any number of input mechanisms, such as a microphone for speech, a touch-sensitive screen for gesture or graphical input, keyboard, mouse, motion input, speech and so forth. An output device 1270 can also be one or more of a number of output mechanisms known to those of skill in the art. In some instances, multimodal systems enable a user to provide multiple types of input to communicate with the computing device 1200. The communications interface 1280 generally governs and manages the user input and system output. There is no restriction on operating on any particular hardware arrangement and therefore the basic features here may easily be substituted for improved hardware or firmware arrangements as they are developed.

For clarity of explanation, the illustrative system embodiment is presented as including individual functional blocks including functional blocks labeled as a “processor” or processor 1220. The functions these blocks represent may be provided through the use of either shared or dedicated hardware, including, but not limited to, hardware capable of executing software and hardware, such as a processor 1220, that is purpose-built to operate as an equivalent to software executing on a general purpose processor. For example, the functions of one or more processors presented in FIG. 12 may be provided by a single shared processor or multiple processors. (Use of the term “processor” should not be construed to refer exclusively to hardware capable of executing software.) Illustrative embodiments may include microprocessor and/or digital signal processor (DSP) hardware, read-only memory (ROM) 1240 for storing software performing the operations discussed below, and random access memory (RAM) 1250 for storing results. Very large scale integration (VLSI) hardware embodiments, as well as custom VLSI circuitry in combination with a general purpose DSP circuit, may also be provided.

The logical operations of the various embodiments are implemented as: (1) a sequence of computer implemented steps, operations, or procedures running on a programmable circuit within a general use computer, (2) a sequence of computer implemented steps, operations, or procedures running on a specific-use programmable circuit; and/or (3) interconnected machine modules or program engines within the programmable circuits. The system 1200 shown in FIG. 12 can practice all or part of the recited methods, can be a part of the recited systems, and/or can operate according to instructions in the recited non-transitory computer-readable storage media. Such logical operations can be implemented as modules configured to control the processor 1220 to perform particular functions according to the programming of the module. For example, FIG. 12 illustrates three modules MOD1 1262, MOD2 1264 and MOD3 1266, which are modules configured to control the processor 1220. These modules may be stored on the storage device 1260 and loaded into RAM 1250 or memory 1230 at runtime or may be stored as would be known in the art in other computer-readable memory locations.

While various examples of the present invention have been described above, it should be understood that they have been presented by way of example only, and not limitation. Numerous changes to the disclosed examples can be made in accordance with the disclosure herein without departing from the spirit or scope of the invention. Thus, the breadth and scope of the present invention should not be limited by any of the above described examples. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular examples only and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

What is claimed is:

1. An apparatus configured to support at least one recording device, comprising:
a base element comprising a horizontal segment and a vertical segment, wherein the vertical segment extends at a midpoint along a length of the horizontal segment;
at least one movable joint connecting an arm to the base element, wherein the arm houses the at least recording one device; and
a receiving element configured to receive user input.

2. The apparatus of claim 1, wherein the at least one recording device comprises either a video recording device or an audio recording device.

3. The apparatus of claim 2, wherein the arm further comprises a screen mounted on the arm, wherein the screen is configured to receive visual data from the video recording device and display the received visual data.

4. The apparatus of claim 2, further comprising at least one light positioned around an exterior edge of the video recording device.

5. The apparatus of claim 1, wherein the at least one recording device comprises at least two video recording devices, wherein each of the at least two video recording devices are configured to record separately.

6. The apparatus of claim 5, wherein a first video recording device of the at least two video recording devices comprises a lens of a first shape and a second video recording device of the at least two video recording devices comprises a lens of a second shape, wherein the first shape and the second shape comprise different shapes.

7. The apparatus of claim 1, wherein the arm further comprises a sensor mounted on the arm, wherein the sensor comprises a microphone configured to receive voice commands.

8. The apparatus of claim 1, further comprising at least one sensor, wherein the sensor is mounted on at least one of the arm and the body, wherein the at least one sensor comprises at least one of an inertial measurement unit sensor configured to receive rotational orientation data and an accelerometer configured to receive gravitational force data.

9. The apparatus of claim 1, further comprising a strap that removably couples to a first end of the horizontal segment and to a second end of the horizontal segment.

10. The apparatus of claim 9, further comprising a sensor, wherein the at least one sensor is mounted on at least one of the body and the strap, wherein the at least one sensor comprises a biological sensor configured to receive physiological data of a user.

11. The apparatus of claim 10, wherein the biological sensor comprises at least one of: a heart rate monitor, a blood flow sensor, a sweat sensor, a respiratory rate monitor, a pressure sensor, a blood pressure monitor, a blood glucose level sensor, an electrocardiogram sensor, a skin conductance sensor, and/or any combination thereof.

12. The apparatus of claim 1, further comprising a coupling element configured to secure the arm against the base element, wherein the coupling element comprises at least one of a magnet, a clasp, a plurality of fasteners, and a male and a female connector element.

13. The apparatus of claim 1, wherein the arm further comprises a removable external housing.

14. The apparatus of claim 1, wherein the base element houses at least one of a processor, a battery, and a wireless communication module.

15. The apparatus of claim 13, wherein the processor is configured to perform at least one of:
cause the at least one recording device to start and stop recording based on at least one of the received voice commands, the received physiological data of the user, and the received user input;
aim at least one light at a target area based on at least one of received location data of the target area, received position and orientation data of the arm, and received user input;
change at least one of a position and an orientation of at least one of the arm and the recording device based on at least one of the received location data of the target area, the received position and orientation of the arm, and the received user input; and
capture and store, at a memory module, a screenshot or video clip from the at least one recording device based on received user input or data from the at least one recording device.

16. The apparatus of claim 13, wherein the wireless communication module is communicatively coupled to an external joystick, and wherein the processor is configured to adjust at least one of a position and an orientation of the arm based on data received from the external joystick.

17. The apparatus of claim 12, wherein the wireless communication module is communicatively coupled to an external display, and wherein the processor is configured to send data received from the at least one recording device to the external display.

18. The apparatus of claim 12, wherein the wireless communication module is communicatively coupled to an external computing device, and wherein the processor is configured to send data received from the at least one recording device to the external computing device and to receive commands from the external computing device.

19. The apparatus of claim 1, further comprising a removable sleeve configured to cover the arm, wherein the removable sleeve comprises a first opening and a second opening configured to receive the at least one recording device.

20. The apparatus of claim 1, wherein the arm further comprises at least one actuator configured to adjust at least one of a position and an orientation of the recording device based on at least one of the received user input and input from at least one sensor housed on the apparatus.
